(19) United States
(12) Patent Application Publication
(10) Pub. No.: US 2026/0007862 A1
(43) Pub. Date: Jan. 8, 2026

(54) NEEDLE HOLDER

(71) Applicant: KAIROS CO., LTD., Daegu (KR)

(72) Inventor: Yong Min KIM, Daegu (KR)

(21) Appl. No.: 18/842,124

(22) PCT Filed: Nov. 21, 2022
(86) PCT No.: PCT/KR2022/018401

§ 371 Date: Mar. 25, 2025

(30) Foreign Application Priority Data
Mar. 31, 2022 (KR) 10-2022-0040656

(51) Int. Cl. A61M 25/06

(52) U.S. Cl. CPC A61M 25/065

(57) ABSTRACT

The present invention relates to a needle holder capable of fixing a catheter and minimizing skin irritation, wherein a blood vessel proximity apparatus can be fixed without skin irritation by means of a body member in which a body part, an injection end, and a discharge end are integrally formed.

### TECHNICAL FIELD

The present invention relates to a needle holder which connects a blood vessel proximity apparatus such as an IV catheter inserted into a patient and an infusion set.

Furthermore, the present invention relates to a needle holder which secures a blood vessel proximity apparatus without skin irritation and secures an elastic Luer lock syringe.

### BACKGROUND ART

Conventionally, there are two types of needle holders used to connect an infusion set to a cannula, that is, a rubber type and a rotary type.

The rubber type is made of a rubber material and is used to secure a needle by combining a male and female set, but has a disadvantage in that female and male parts are likely to fall out easily.

In contrast, the rotary type adopts a method of joining to a circular screw thread and has good securing power, but has the disadvantage of causing irritation to the patient's skin when used for a long time due to its round and protruding shape.

Meanwhile, Korean Utility Model Publication No. 20-2019-0001950 (Fixing means for IV catheter) relates to an IV catheter fixing member for fixing a medical tube (infusion line), and discloses
a fixing means for IV catheter, which includes an attachment fixing part, which is attached to the skin of a patient and fixes a catheter connected to a needle; and a tube fixing part, which is connected to the attachment fixing member so as to fix an infusion line connected to the catheter,
wherein the tube fixing part includes:
a receiving part, which is fixed to the attachment fixing part and has an open upper portion;
a plurality of support grooves, which are formed in the receiving part and formed in the longitudinal direction where the infusion line is coupled so as to support the infusion line;
an opening/closing part, which is connected to the receiving part so as to open/close the open upper portion of the receiving part; and
a support protrusion, which is formed in the opening/closing part and comes in close contact with and supports the infusion line supported by the support grooves at the time of closing.

In addition, Korean Patent Publication No. 10-2017-0056903 (IV catheter) discloses an IV catheter, including:
a cup-shaped catheter hub, which has a catheter tube installed at a front tip and a first space with an open rear end;
a valve unit, which is installed in the first space and blocks a fluid flowing in a first direction from a front end of the first space to the rear end through the catheter tube; and
a sliding unit, which is installed in the first space so as to slide in the first direction or a second direction opposite to the first direction and opens/closes the valve unit according to the sliding,
wherein the valve unit is arranged between the front end of the first space and the sliding unit in the first space.

Furthermore, Korean Patent Publication No. 10-2017-0102918 (IV catheter system) discloses an IV catheter system including:
a catheter hub (100), which includes a tubular catheter (101) attached to a catheter hub body (102) at a proximal end and having a lumen, and catheter hub cavities (103, 107) in fluid communication with the lumen of the tubular catheter (101);
a needle hub (200), which includes a needle (201) extending distally from a needle hub body (202), the needle (201) having an expansion portion (204) and a high friction surface portion (207) at its distal end region;
a needle shield (300), which includes at least one elastic arm (301) extending distally from a base plate (302), the base plate (302) having a through hole for receiving the needle (201),
wherein the needle hub (200) is arranged within the catheter hub (100) so that the needle (201) is arranged so as to slide through the lumen of the catheter (101) and thus the needle (201) can be withdrawn proximally from the catheter hub (100);
wherein the needle shield (300) is arranged within the catheter hub cavities (103, 107) in such a way that the needle shield (300) is held on the needle (201) through cooperation between the needle shield (300) and an inner wall of the catheter hub (100) within the catheter hub cavities (103, 107), so that the at least one arm (301) is placed on the needle (201) and elasticity is imparted by the needle (201), and the needle (201) is slidably arranged within the through hole of the base plate (302) in an assembled state; and
wherein when the needle hub (200) is withdrawn from the catheter hub (100) so as to release the needle shield (300) from the catheter hub (100), outer diameter of the needle expansion portion (204) is larger than inner diameter of the through hole of the base plate (302), and the at least one arm (301) covers a tip of the needle (201) in a released state.

### PRIOR ART DOCUMENTS

### Patent Documents

(Patent Document 1) Korean Utility Model Publication No. 20-2019-0001950 (Fixing means for IV catheter)
(Patent Document 2) Korean Patent Publication No. 10-2017-0056903 (IV catheter)
(Patent Document 3) Korean Patent Publication No. 10-2017-0102918 (IV catheter system)

### DETAILED DESCRIPTION OF THE INVENTION

### Technical Problems

The technical problem that this invention seeks to solve is to provide a needle holder which can secure a catheter and minimize skin irritation.

The technical problem that this invention seeks to solve is to provide a connector, which is connected in a female Luer lock manner and has strong fixing force and elasticity without causing irritation.

### Problem Solving Means

In order to achieve the above purposes, a needle holder according to the present invention includes a body member and a fixing member.

A body member (100) includes a body part (110), an injection end (120), a discharge end (130), and a side injection port (140).

The body part (110) connects the injection end (120) that is coupled to an infusion set and the discharge end (130) that is coupled to a needle, in the middle of the injection end (120) and the discharge end (130), and is attached to the skin of a patient so as to inject fluid or medication from the infusion set into a needle through a through hole (150) that penetrates the inside of the body part (110).

The injection end (120) is a cylindrical end that can be connected to an IV line of the infusion set, and the through hole (150) formed inside the body part (110) is extended through the injection end (120).

A large-diameter injection end (121) is formed large enough to allow an IV line (123) to be fitted to an inner portion of the injection end (120), and a small-diameter injection end (124) is formed small enough to allow the IV line (123) to be fitted to an outer portion of the injection end (120).

The large-diameter injection end (121) may have an internal step (122) formed at a connection portion with the body part (110), and the IV line (123) can be inserted and connected up to such an internal step (122) portion.

The discharge end (130) is a cylindrical end, into which a needle hub can be fitted, and the through hole (150) of the body part (110) is extended through the discharge end (130).

The through hole (150) is formed through the injection end (120), the body part (110) and the discharge end (130), so as to function as a passage for supplying medicine supplied from the infusion set to the needle.

The side injection port (140) formed in one side surface of the body part (110) can provide a function that allows an injection needle to be inserted without removing a connecting part, if additional medicine needs to be directly administered into a human body in the process of injecting a fluid into the human body.

A fixing member (200) includes elastic members (210), supports (220), grip members (230), and handles 240.

The elastic members (210) are formed between both side surfaces of the discharge end (130) and the supports (220) of the fixing member (200), so that the grip members (230) fix the needle hub to be connected to the discharge end (130) by elastic force.

When the handles (240) of the fixing member (200) are pressed, the grip members (230) attached to ends of the supports (220) are separated from the discharge end (130), and at this time, the needle hub is inserted into and coupled to the discharge end (130), wherein if the pressed handles (240) are released after the needle hub is fully inserted into the grip members (230), the needle hub is securely fixed by the grip members (230) and is prevented from coming off.

The supports (220) are attached to ends of the elastic members (210) so as to form a frame of the fixing member (200), and have handles formed on ends at one side and grip members (230) formed on ends at the other side. The supports (220) are formed to be gradually inclined so that the ends at the handle (240) side are positioned further outward and distance between the handles (240) and the body part (110) is greater than distance from the body part (110) at connection portions of the elastic members (210), thereby making it easy to hold the handles (240).

The grip members (230) are formed as sawtooth-shaped protrusions at the ends of the supports (220) so as to press and fix the needle hub, wherein a needle hub flange is caught on the sawtooth-shaped protrusions, thereby being prevented from being separated from the discharge end (130). A plurality of sawtooth-shaped protrusions may be formed depending on the structure of a needle hub to be joined.

The handles (240) are formed at the ends of the supports, so that by pressing the handles (240), the grip members (230) on the other side are separated from the discharge end (130).

Preferably, reinforcing ribs (250) may be further provided on outer portions of the supports (220).

The reinforcing ribs (250) are attached to outer surfaces of the supports (220) in the longitudinal direction of the supports (220) so as to supplement the rigidity of the supports.

### Effect of the Invention

As described above, the present invention can provide elasticity to the fixing member of the needle holder, thereby strengthening fixing force for the needle hub through the grip members by the elasticity.

In addition, it is possible to provide a female Luer lock type connector, which fixes a catheter, minimizes skin irritation, and has strong fixing force and elasticity.

The existing rubber-type needle holder made of a rubber material had a disadvantage in that the male and female set is likely to be detached from each other. However, according to the present invention, it is expected that the combination of the main body member and the fixing member will have the effect of stably fixing the needle hub.

In addition, the shape of the existing rotary type needle holder was round and protruding, so there was a disadvantage in irritating the patient's skin when used for a long time. However, according to the present invention, the body member and fixing member are formed to minimize skin irritation, thereby resolving the disadvantages of the existing needle holder.

### BRIEF DESCRIPTION OF THE DRAWINGS

FIG. 1 and FIG. 2 are perspective views of a needle holder according to the present invention.

FIG. 3 is a plan view of a needle holder according to the present invention.

FIG. 4 and FIG. 5 are cross-sectional views of a needle holder according to the present invention.

FIG. 6 shows an embodiment of a needle holder according to the present invention.

### BEST MODE FOR CARRYING OUT THE INVENTION

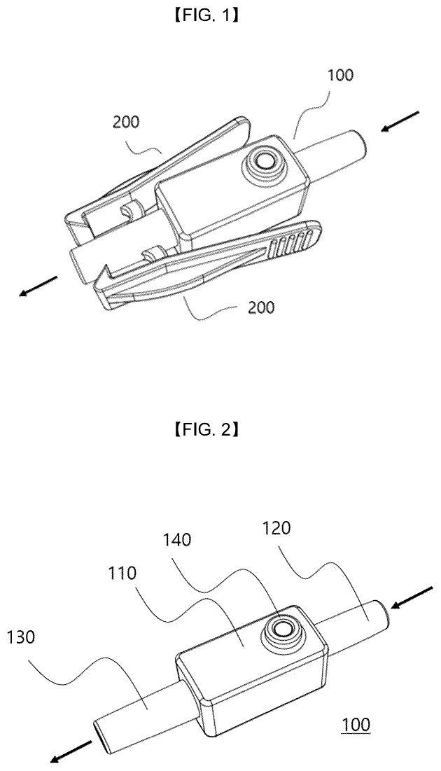

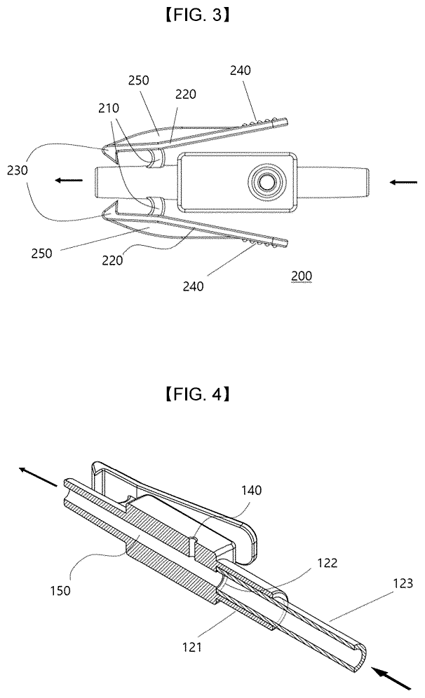

Hereinafter, embodiments of the present invention will be explained with reference to the accompanying drawings FIG. 1 to FIG. 6.

A needle holder according to the present invention includes a body member 100 and a fixing member 200 attached in a gripper shape to both side surfaces of the body member (100).

The body member (100) includes a body part (110), an injection end (120), a discharge end (130), and a side injection port (140).

The body part (110) connects the injection end (120) that is coupled to an infusion set and the discharge end (130) that is coupled to a needle, in the middle of the injection end (120) and the discharge end (130), and is attached to the skin of a patient so as to inject fluid or medication from the infusion set into a needle through a through hole (150) that penetrates the inside of the body part (110).

The injection end (120) is a cylindrical end that can be connected to an IV line of the infusion set, and the through hole (150) formed inside the body part (110) is extended through the injection end (120).

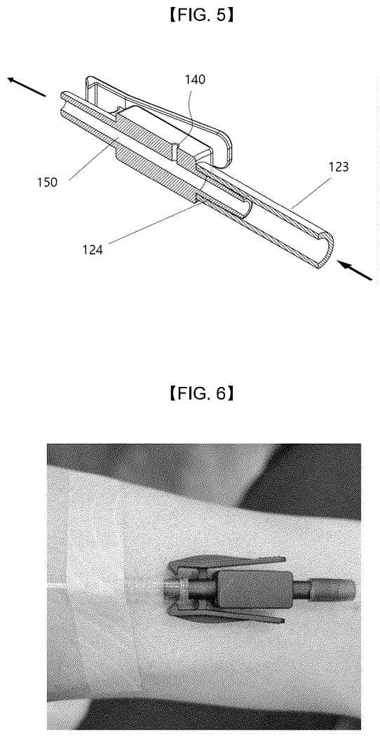

As shown in FIG. 4, a large-diameter injection end (121) is formed large enough to allow an IV line (123) to be fitted to an inner portion of the injection end (120), and
as shown in FIG. 5, a small-diameter injection end (124) is formed small enough to allow the IV line (123) to be fitted to an outer portion of the injection end (120).

The large-diameter injection end (121), as shown in FIG. 4, may have an internal step (122) formed at a connection portion with the body part (110), and the IV line (123) can be inserted and connected up to such an internal step (122) portion.

The discharge end (130) is a cylindrical end, into which a needle hub can be fitted, and the through hole (150) of the body part (110) is extended through the discharge end (130).

The through hole (150) is formed through the injection end (120), the body part (110) and the discharge end (130), so as to function as a passage for supplying medicine supplied from the infusion set to the needle.

The side injection port (140) formed in one side surface of the body part (110) can provide a function that allows an injection needle to be inserted without removing a connecting part, if additional medicine needs to be directly administered into a human body in the process of injecting a fluid into the human body.

The fixing member (200) includes elastic members (210), supports (220), grip members (230), and handles 240.

The elastic members (210) are formed between both side surfaces of the discharge end (130) and the supports (220) of the fixing member (200), so that the grip members (230) fix the needle hub to be connected to the discharge end (130) by elastic force.

When the handles (240) of the fixing member (200) are pressed, the grip members (230) attached to ends of the supports (220) are separated from the discharge end (130), and at this time, the needle hub is inserted into and coupled to the discharge end (130), wherein if the pressed handles (240) are released after the needle hub is fully inserted into the grip members (230), the needle hub is securely fixed by the grip members (230) and is prevented from coming off.

The supports (220) are attached to ends of the elastic members (210) so as to form a frame of the fixing member (200), and have handles (240) formed on ends at one side and grip members (230) formed on ends at the other side. The supports (220) are formed to be gradually inclined so that the ends at the handle (240) side are positioned further outward and distance between the handles (240) and the body part (110) is greater than distance from the body part (110) at connection portions of the elastic members (210), thereby making it easy to hold the handles (240).

The grip members (230) are formed as sawtooth-shaped protrusions at the ends of the supports (220) so as to press and fix the needle hub, wherein a needle hub flange is caught on the sawtooth-shaped protrusions, thereby being prevented from being separated from the discharge end (130). A plurality of sawtooth-shaped protrusions may be formed depending on the structure of a needle hub to be joined.

The handles (240) are formed at the ends of the supports, so that by pressing the handles (240), the grip members (230) on the other side are separated from the discharge end (130).

Preferably, reinforcing ribs (250) may be further provided on outer portions of the supports (220).

The reinforcing ribs (250) are attached to outer surfaces of the supports (220) in the longitudinal direction of the supports (220) so as to supplement the rigidity of the supports.

The operation method of the needle holder is explained with reference to FIG. 6 which shows a usage status.

An assembly of a needle and a needle hub is fitted into the discharge end of the needle holder according to the present invention.

The needle hub is fitted into and coupled to the discharge end, wherein the handles of the fixing member are pressed to open the grip members, the flange of the needle hub is sufficiently inserted, and then the handles of the fixing member which are being pressed are released so that the grip members fix the needle hub with the elastic force of the elastic members. At this time, the sawtooth-shaped protrusions of the grip members are held on the flange, thereby fixing the needle hub so that the needle hub cannot come off from the needle holder.

Conversely, in order to separate the needle from the needle holder, the handles are pressed to separate the grip members from the needle hub and the needle hub is separated from the discharge end, so that the needle can be removed.

An IV line is inserted into the injection end of the needle holder of the present patented invention.

If the injection end is the large-diameter injection end (121), the IV line can be coupled by being fitted into the through hole of the injection end, and if the injection end is the small-diameter injection end (123), the IV line can be coupled by being fitted onto the outer surface of the injection end.

As described above, according to the present invention, the fixing member of the needle holder is provided with elasticity, thereby reinforcing the fixing force for the needle hub through the grip members and minimizing skin irritation.

1: A needle holder capable of fixing a catheter and minimizing skin irritation, characterized in that a blood vessel proximity apparatus can be fixed without skin irritation by means of a body member in which a body part, an injection end, and a discharge end are integrally formed.

2: The needle holder according to claim 1, characterized in that the body part is attached to the skin of a patient so as to inject fluid or medication from the infusion set into a needle through a through hole that penetrates the inside of the body part.

3: The needle holder according to claim 1, characterized in that the injection end is a cylindrical end capable of being connected to an IV line of the infusion set, and the through hole formed inside the body part is extended through the injection end.

4: The needle holder according to claim 1, characterized in that the discharge end is a cylindrical end capable of fitting into a needle hub, and the through hole of the body part is extended through the discharge end.

5: The needle holder according to claim 1, characterized in that a blood vessel proximity apparatus can be fixed without skin irritation, further comprising a fixing member including elastic members, supports, grip members, and handles.

6: The needle holder according to claim 5, characterized in that the elastic members are formed between both side surfaces of the body part and the supports of the fixing member, and when the handles of the fixing member are pressed, the grip members attached to ends of the supports are separated from the discharge end, and needle hub inserted into the discharge end is securely fixed by the grip members.

7: The needle holder according to claim 5, characterized in that the supports are attached to ends of the elastic members so as to form a frame of the fixing member, and have handles formed on ends at one side and grip members formed on ends at the other side, and the supports are formed to be gradually inclined so that the ends at the handle side are positioned further outward and distance between the handles and the body part is greater than distance from the body part at connection portions of the elastic members.

8: The needle holder according to claim 5, characterized in that the grip members are formed as sawtooth-shaped protrusions at the ends of the supports so as to press and fix the needle hub, wherein a needle hub flange is caught on the sawtooth-shaped protrusions, thereby being prevented from being separated from the discharge end.

9: The needle holder according to claim 5, characterized in that the handles are formed at the ends of the supports, so that by pressing the handles, the grip members on the other side are separated from the discharge end.
